(19) United States
(12) Patent Application Publication
(10) Pub. No.: US 2025/0104127 A1
(43) Pub. Date: Mar. 27, 2025

(54) SYSTEMS AND METHODS FOR SKIN BIOMOLECULAR PROFILE ASSESSMENT USING ARTIFICIAL INTELLIGENCE

(71) Applicant: VANDERBILT UNIVERSITY, Nashville, TN (US)

(72) Inventors: Christina C. MARASCO, Nashville, TN (US); Stacy D. SHERROD, Nashville, TN (US); Amelia TAYLOR, Nashville, TN (US); Donovan TAYLOR, Nashville, TN (US)

(21) Appl. No.: 18/730,913

(22) PCT Filed: Jan. 20, 2023
(86) PCT No.: PCT/US2023/011249

§ 371 Date: Jul. 22, 2024

Related U.S. Application Data
(60) Provisional application No. 63/301,271, filed on Jan. 20, 2022.

(51) Int. Cl. G06Q 30/0601; G16H 10/20; G16H 20/40

(52) U.S. Cl. CPC G06Q 30/0631; G16H 10/20; G16H 20/40

(57) ABSTRACT

Skin biomolecular profile assessment methods and systems that can analyze the molecular composition of the skin using molecular-level, user-specific data to assess an individual's skin state and/or disease state are described herein. An example method includes receiving skin data associated with a subject, where the skin data includes a biomolecular profile. The method also includes inputting the skin data into a trained artificial intelligence (AI) model and receiving, from the trained AI model, a skin care prediction.

### CROSS-REFERENCE TO RELATED APPLICATIONS

This application claims the benefit of U.S. provisional patent application No. 63/301,271, filed on Jan. 20, 2022, and titled “Skin Biomolecular Profiling kit, Assessment, and Skin-Product Fit Algorithm,” the disclosure of which is expressly incorporated herein by reference in its entirety.

### BACKGROUND

Biomolecular skin assessment is a process of analyzing the molecular composition of the skin to determine skin phenotypes, health, and/or identify potential health issues such as disease. Biomolecular skin assessments may be performed using various modalities known in the art such as microscopy, spectroscopy, and bioimpedance analysis. It would be desirable to provide a system and method that uses the results of biomolecular skin assessments to develop personalized skin care recommendations. Additionally, a system and method for providing personalized skin care recommendations in an efficient and effect manner is needed. The present disclosure addresses these needs.

### SUMMARY

A skin biomolecular profile assessment method and system that can analyze the molecular composition of the skin using molecular-level, user-specific data to assess an individual's skin state(s) and/or disease state(s) and drive an artificial intelligence (AI)-based recommendation engine to provide skin care recommendations are described herein.

An example computer-implemented method for skin profile assessment is described herein. The method includes receiving skin data associated with a subject, where the skin data includes a biomolecular profile. The method also includes inputting the skin data into a trained artificial intelligence (AI) model and receiving, from the trained AI model, a skin care prediction.

Additionally, the biomolecular profile includes molecular analysis data. Optionally, the molecular analysis data is mass spectrometry data.

Alternatively or additionally, the biomolecular profile includes a plurality of biomarkers. For example, each of the biomarkers is associated with at least one skin state or at least one disease. In some implementations, the method further includes selecting one or more of the biomarkers from the biomolecular profile, and the step of inputting the skin data into the trained AI model includes inputting the selected one or more of the biomarkers into the trained AI model. Optionally, the selected one or more of the biomarkers are the top-n biomarkers predictive of the skin care prediction.

Alternatively or additionally, the skin data further includes user-reported data. Optionally, the user-reported data includes at least one of an allergy, a sensitivity, a skin type, a product/ingredient preference, or a product/ingredient usage information.

Alternatively or additionally, the skin care prediction includes at least one of a product recommendation, an ingredient recommendation, a dietary recommendation, a lifestyle recommendation, or a skin insight.

Alternatively or additionally, the trained AI model is a machine learning model. In some implementations, the machine learning model is a supervised machine learning model. In some implementations, the machine learning model is a deep learning model.

An example method of treatment is also described herein. The method includes obtaining a skin care prediction for a subject using a computer-implemented method as described herein. The method also includes treating the subject according to the skin care prediction.

An example system for skin profile assessment is also described herein. The system includes an artificial intelligence (AI) model, and a processor and a memory, the memory having computer-executable instructions stored thereon. The processor is configured to input skin data associated with a subject into the AI model, where the skin data includes a biomolecular profile. The processor is also configured to receive, from the AI model, a skin care prediction.

Another computer-implemented method for skin profile assessment is also described herein. The method includes receiving, by one or more processors, a mass spectrometry data set of a subject (e.g., person or animal); applying, by the one or more processors, the mass spectrometry data set to an analysis employing skin biomolecular profile features derived from one or more trained machine learning models, wherein the skin biomolecular profile features are linked to one of a plurality of skin care or treatment ingredients and/or product; and outputting, by the one or more processors, an AI-derived output comprising at least one of the plurality of skin care or treatment ingredients or products based on the analysis.

Alternatively or additionally, one or more of the skin biomolecular profile features are linked to a skin biomarker that includes at least one of overall skin health score, skin type score, skin structure score, skin function score, skin hydration score, skin sensitivity score, age, and appearance score.

Alternatively or additionally, the method further includes performing a mass spectrometry analysis to generate the mass spectrometry data set. Optionally, the mass spectrometry analysis is performed using at least one of liquid chromatography-mass spectrometry analysis and/or laser desorption/ionization mass spectrometry analysis.

Alternatively or additionally, the skin biomarker includes one of: amino acids, organic acids, acylcarnitines (e.g., hexadecenoyl carnitine, m/z 398.327), ceramides, fatty acids (sapienic acid, m/z 254.225), bile acids (glycocholic acid, m/z 466.316).

Alternatively or additionally, the method further includes receiving, by the one or more processors, a request for a sample collection kit through a user portal; and generating, by the one or more processors, a work order for a shipment to the sample collection kit to an address and user using information associated with a user collected from the user portal.

Alternatively or additionally, the method further includes generating, via a recommendation engine, a first ingredients or a skincare product recommendation using the AI-derived output and a second ingredients or a skincare product recommendation using the AI-derived output in combination with a user provided parameter. Optionally, the user provide parameter includes (i) User climate and lifestyle, (ii) known allergies/sensitivities, (iii) known skin type and skin issue information, (iv) preferences regarding price, marketed features (e.g., chemical-free, organic, sustainability), and unmarketed features (e.g., texture, scent), routine difficulty (number of products used), and/or (v) current products and brand preferences; (b) molecular assessment results of user-supplied skin sample; (c) existing product data such as (i) ingredients, (ii) metadata (e.g., brand, price, product category, marketed features), (iii) reviews (e.g., unmarketed features); (d) an ingredient knowledge database containing ingredient classes and properties based on known interactions with skin and/or structure/function relationships; and (e) skin knowledgebase containing skin biomolecule classes, properties, and ranges of amounts corresponding to skin biomolecular profiles.

Alternatively or additionally, the method further includes applying, by the one or more processors, the mass spectrometry data set to a second analysis employing skin biomolecular profile features derived from one or more trained machine learning models linked to one of a plurality one of pharmaceutical treatments and/or skin disease/condition states; and outputting, by the one or more processors, a second AI-derived output comprising at least one of the plurality of pharmaceutical treatments and/or skin disease/condition states based on the second analysis. Optionally, the second AI-derived output includes one of: a skin cancer score, a rosacea score, an eczema score, atopic dermatitis score, and/or a seborrheic dermatitis score.

In some implementations, the skin biomolecular profile features include a first skin biomolecular profile feature associated with a retention time alignment indication of the mass spectrometry data set.

In some implementations, the skin biomolecular profile features include a second skin biomolecular profile feature associated with a peak picking indication of the mass spectrometry data set.

In some implementations, the skin biomolecular profile features include a third skin biomolecular profile feature associated with a deconvolution indication of the mass spectrometry data set.

In some implementations, the skin biomolecular profile features include a fourth skin biomolecular profile feature associated with an annotation indication of the mass spectrometry data set.

Alternatively or additionally, the one or more trained machine learning models include one of a regularized linear regression model, a gradient boosted decision tree model, a support vector machine model, and a neural network.

Alternatively or additionally, the method further includes performing, by the one or more processors, a sentiment analysis using product reviews to assess positive or negative sentiment regarding a product; and performing, by the one or more processors, semi-supervised learning based on the sentiment analysis.

Alternatively or additionally, the method further includes performing, by the one or more processors, statistical analysis of metadata and quantitative product reviews to assess general product perception and quality; and performing, by the one or more processors, semi-supervised learning based on the statistical analysis.

Alternatively or additionally, the method further includes performing, by the one or more processors, natural language processing analysis to identify keywords related to unmarketed features (e.g., texture, scent, absorption, stickiness) within product reviews to tag products based on these features for matching to user preferences; and performing, by the one or more processors, semi-supervised learning based on the natural language processing analysis.

Alternatively or additionally, the method further includes performing, by the one or more processors, variation Autoencoder Clustering or cosine similarity analysis to compare product compositions; and performing, by the one or more processors, semi-supervised learning based on the variation Autoencoder Clustering or cosine similarity analysis.

Optionally, semi-supervised learning is one of random forest analysis, multivariate regression analysis, or neural network analysis.

An example kit for collecting samples is also described herein. The kit includes an adhesive substrate to collect a sample comprising outer layers of the skin of a user; and a labeled collection enclosure (e.g., a cardboard tube holder or a cutout for a kit material) having a label associated with the user.

Additionally, the kit optionally further includes a second adhesive substrate to collect a second sample of the user, wherein the labeled collection enclosure includes an insert for each of the adhesive substrate and second adhesive substrate.

Alternatively or additionally, the kit optionally further includes an applicator configured to apply the adhesive at a consistent pressure (e.g., roller, foam block).

Alternatively or additionally, the kit optionally further includes a cleaning kit item comprising at least one of: alcohol wipe, micellular water wipe, or mild face wash; and a substrate removal kit item comprising at least one of tweezers, tabs, or gloves.

In some embodiments, the system includes a sample collection kit that allows a user to painlessly collect a sample of their epidermis (and any exogenous deposits on the epidermis) either using a swab or adhesive approach that is resistant to user error. The kit is shipped directly to the user with return packaging. The system also includes an analysis system for broadscale molecular analysis of specific skin biomarkers linked to skin biomolecular profile (e.g., overall skin health, skin type, structure, function, hydration, sensitivity, age, and appearance), disease state (e.g., atopic dermatitis, rosacea), or exposure (e.g., xenobiotics) using liquid chromatography-mass spectrometry (LC-MS) and/or matrix-assisted laser desorption/ionization mass spectrometry (MALDI-MS) methods. Non-exhaustive examples of skin biomarkers include molecularly diverse targets such as amino acids, acylcarnitines (hexadecenoyl carnitine, m/z 398.327), ceramides, fatty acids (sapienic acid, m/z 254.225), bile acids (glycocholic acid, m/z 466.316). The system also includes a data analysis system to perform skin fit artificial intelligence that matches the findings from the molecular analysis to existing over-the-counter skincare products, pharmaceutical treatments, or skin disease/condition states.

Over-the-counter and prescription skincare product matches can be based on (a) User-reported inputs comprising (i) User climate and lifestyle, (ii) known allergies/sensitivities, (iii) known skin type and skin issue information, (iv) preferences regarding price, marketed features (e.g., chemical-free, organic, sustainability), and unmarketed features (e.g., texture, scent), skincare routine difficulty (e.g., number of products used), and/or (v) current products and brand preferences; (b) molecular assessment results of user-supplied skin sample; (c) existing product data such as (i) ingredients, (ii) metadata (e.g., brand, price, product category, marketed features), (iii) reviews (e.g., unmarketed features); (d) an ingredient knowledge database containing ingredient classes and properties based on known interactions with skin and/or structure/function relationships; and (e) skin knowledgebase containing skin biomolecule classes, properties, and ranges of amounts corresponding to skin biomolecular profiles. The system is configured to convert a user's skin biomolecular profile result to customer-specific skin insights and product (and/or ingredient) guidance through the skin fit AI. This AI links the skin biomolecular profile results (biomolecules and relative amounts) to skin insights, such as skin type, hydration level, and barrier function integrity. These insights are delivered directly to the user via a skin report and also are used to match to the ingredient knowledgebase, generating a list of ideal ingredients for the user as well as ingredients to avoid. For instance, for a user with lower levels of stratum corneum lipids and proteins (a result that is captured through the molecular assay or analysis), a synthetic detergent would be more suited to their skincare needs as synthetic detergents leave lipids and proteins of the stratum corneum (epidermis) in place, causing less irritation; whereas, traditional soaps tend to strip these molecules (Levin and Miller 2011). The ideal ingredients are then mapped to the product ingredients, metadata, and review databases to determine product-level matches in the categories (cleanser, moisturizer, serum, etc.), price ranges, and marketed and unmarketed feature sets reported by the user. User-reported climate, lifestyle, skin type, issues, and allergies/sensitivities will be compared to the skin insights and product/ingredient recommendations to ensure any potential product or ingredient recommendations have included the user-reported preferences. The skin fit AI can deliver the customer-specific skin insights and product (and ingredient) guidance directly to the customer profile using a web/mobile application.

The system includes a user report that can provide both approachable yet scientific skin characteristics, insights, and product guidance to individuals to allow them to make informed choices. By providing product guidance only within the product categories (e.g., cleansers, moisturizers, serums) and price points of interest to the customer, the system can deliver a solution that scientifically addresses their skincare needs, routine preferences, and spending targets.

The example system and corresponding services can provide individuals with a deeper, more accurate understanding of their unique skin (via biomolecular profile assessment) and their skincare needs (e.g., for over-the-counter skincare products), particularly when their skin changes, e.g., from aging, diet, hormones, environmental factors, or underlying disease state.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram illustrating an artificial intelligence (AI) model operating in inference mode according to an implementation described herein.

FIG. 2 is a flowchart illustrating example operations for skin biomolecular profile assessment according to an implementation described herein.

FIG. 3 is an example computing device.

FIG. 4 illustrates a process overview of the skin assessment platform according to an example described herein.

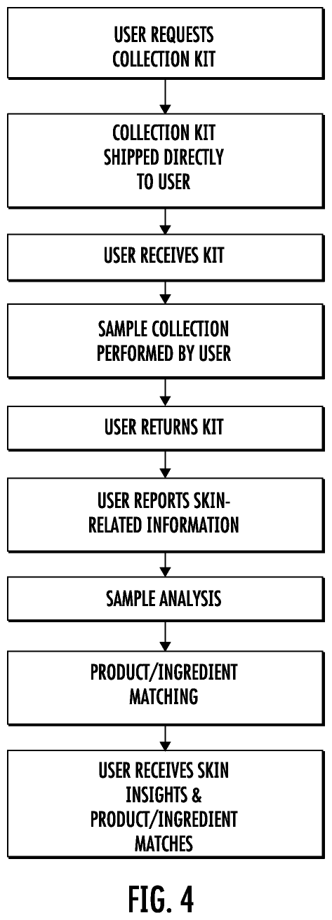

FIG. 5 illustrates an example sample collection kit that can allow a user to collect skin and return it for analysis.

FIG. 6 illustrates an example skin assessment workflow that includes: sample quality validation, sample preparation, followed by LC-MS/MS or LC-IM-MS/MS, data processing, and data analytics.

FIG. 7 illustrates an example data analytics workflow for the data analytics pipeline.

FIG. 8A illustrates example biomarkers exhibiting low and high normality (left and middle, respectively) and normality values of all biomarkers (right). FIG. 8B illustrates example biomarkers exhibiting low and high distribution shifts (left and middle, respectively) and mean shift values of all biomarkers for acne-prone skin state (right).

FIG. 9 illustrates example skin assessment biomolecular profile results for acne-prone skin.

FIG. 10 illustrates the prediction accuracy of a study attained by using single features (labeled 1010) in Logistic Regression compared to mean test prediction accuracy of random forest models using combined features (top right, labeled 1020).

FIGS. 11A-11C illustrate biomolecular profile data outputs specific to a user-reported data (gender (FIG. 11A), age (FIG. 11B), and acne-prone skin state (FIG. 11C).

FIG. 12 is a diagram of an example implementation of the SkinFit AI model including a big data-powered artificial intelligence pipeline.

FIG. 13 is Table 1, which is a list of example inputs and outputs for the skin biomolecular profiling method.

FIG. 14 illustrates an example workflow of the Skin Fit AI model.

FIG. 15 illustrates an example implementation of the Skin Fit AI model.

FIG. 16 is Table 2, which is a list of example analyses.

FIG. 17 illustrates an example output of the skin biomolecular profile assessment platform through a web portal or a printed report.

FIG. 18 illustrates an example report that can be generated using the Skin Fit AI model.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

A skin biomolecular profile assessment method and system that can analyze the molecular composition of the skin using molecular-level, user-specific data (e.g., via mass spectrometry based broadscale molecular analysis) to assess an individual's skin state(s) and/or disease state(s) and drive an artificial intelligence (AI)-based recommendation engine to direct individuals to appropriate over-the-counter products or ingredients, prescription treatments, dietary/lifestyle changes, and supplementation are described herein.

As a true diagnostic technology, the systems and methods described herein can identify clinical skin conditions (e.g., skin cancer, rosacea, eczema, seborrheic dermatitis) based on biomarkers specific to these conditions, providing a less painful (compared to biopsy) or more direct (diagnosis by removing other possibilities) path to a diagnosis. Treatment and supportive skin health products (e.g., over-the-counter and prescription) can be recommended using the system and method described herein. The system and method can be applied individually to skin and hair or in combination thereof.

The systems and methods described herein provide broadscale molecular analyses coupled to advanced data analytics employing artificial intelligence (AI) and/or machine learning (ML) strategies provide a powerful tool to determine biomarkers, yielding critical information on specific skin states, disease states, nutrition status, skin microbiome, pharmaceutical use, and environmental exposure. The detection of levels of biomarkers in a sample from a subject, particularly a skin sample, allow for the determination of one or more underlying biochemical or biophysical deficiencies that causes or is associated with the skin condition or deficiency or aging, e.g., skin fragility, aberrant pigmentation, such as pigmentation loss or excess pigmentation, tendency to shear and the like. Accordingly, the systems and methods described herein provide for a technical solution that aids in the diagnosis and treatment of aging skin, or of a skin condition or deficiency, at a level that exceeds mere visual or tactile inspection by a medical practitioner, e.g., a physician or dermatologist, or a clinician. For example, the systems and methods described herein provide for robust sample collection (e.g., using a skin sample collection kit) and data normalization to account for potential sources of error such as environmental, experimental, and instrument variation. Additionally, because it is difficult to differentiate between different skin states, diseases, outcomes, etc. using conventional methodologies for analyzing biomolecular data, the systems and methods described herein use feature engineering to identify which biomarkers are predictive of various skin states, diseases, outcomes, etc. The selected or identified biomarkers (e.g., a subset of biomarkers) are then used as input to AI models trained for inference. The systems and methods described herein therefore provide improved predictive capabilities over conventional technologies. Moreover, the systems and methods described herein collect data and metadata collected from a wide variety of sources that include, but are not limited to, a plurality of subjects, literature, and products/ingredients to create a data repository from which AI models can be trained to infer connections between a given biomolecular profile (and optionally user data) and desired target (e.g., product/ingredient information, disease state).

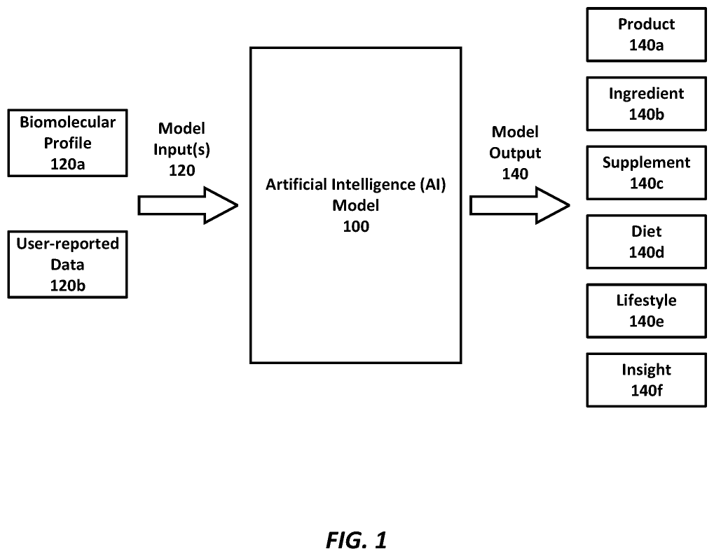

Referring now to FIG. 1, a block diagram illustrating an artificial intelligence (AI) model 100 is shown. In FIG. 1, the AI model 100 is operating in inference mode. The AI model 100 has therefore been trained with a data set (or “dataset”) and is configured to make predictions based on new input data. Accordingly, such a model is sometimes referred to herein as a “trained AI model” or a “deployed AI model.” Optionally, the AI model 100 is a machine learning model. For example, in some implementations, the AI model 100 is a supervised machine learning model. Supervised machine learning models include, but are not limited to, logistic regression models, decision trees, support vector machines, and artificial neural networks. It should be understood that logistic regression models, decision tree models, support vector machines, and artificial neural networks are provided only as example supervised machine learning models. This disclosure contemplates that the supervised machine learning model can be any supervised learning model. Additionally, it should be understood that supervised machine learning models are provided as an example. This disclosure contemplates that the machine learning model may be a semi-supervised or unsupervised learning model.

As described above, a supervised machine learning model “learns” a function that maps an input 120 (also known as feature or features) to an output 140 (also known as target or targets) during training with a labeled data set. Machine learning model training is discussed in further detail below. In some implementations, a trained supervised machine learning model is configured to classify the input 120 into one of a plurality of target categories (i.e., the output 140). In other words, the trained model can be deployed as a classifier. In other implementations, a trained supervised machine learning model is configured to provide a probability of a target (i.e., the output 140) based on the input 120. In other words, the trained model can be deployed to perform a regression.

Optionally, in some implementations, the AI model 100 is a logistic regression (LR) model. An LR model is a supervised learning model that uses the logistic function to predict a target. This disclosure contemplates that the LR model can be implemented using a computing device (e.g., a processing unit and memory as described herein). LR models can be used for classification and regression tasks. LR models are trained with a data set to maximize or minimize an objective function, for example a measure of the LR model's performance (e.g., error such as L1 or L2 loss), during training. This disclosure contemplates that any algorithm that finds the maximum or minimum of the objective function can be used. Additionally, the LR model is optionally a regularized LR model. Regularization is a technique known in the art to address model overfitting. LR models are known in the art and are therefore not described in further detail herein.

Optionally, in some implementations, the AI model 100 is a decision tree (DT) model. An DT model is a supervised learning model that uses a hierarchal tree structure including a root node, branches, internal nodes, and leaf nodes to predict a target. This disclosure contemplates that the DT model can be implemented using a computing device (e.g., a processing unit and memory as described herein). DT models can be used for classification and regression tasks. DT models are trained with a data set to maximize or minimize an objective function, for example a measure of the DT model's performance, during training. Additionally, the DT model is optionally a gradient boosted DT model. Gradient boosting is a technique known in the art for optimization. DT models are known in the art and are therefore not described in further detail herein.

Optionally, in some implementations, the AI model 100 is a support vector machine (SVM). An SVM is a supervised learning model that uses statistical learning frameworks to predict the probability of a target. This disclosure contemplates that the SVM can be implemented using a computing device (e.g., a processing unit and memory as described herein). SVMs can be used for classification and regression tasks. SVMs are trained with a data set to maximize or minimize an objective function, for example a measure of the SVM's performance, during training. SVMs are known in the art and are therefore not described in further detail herein.

Optionally, in some implementations, the AI model 100 is an artificial neural network (ANN). Optionally, the ANN is a deep neural network. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanH, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation. ANNs are known in the art and are therefore not described in further detail herein.

As described above, the AI model 100 is trained to map the input 120 to the output 140. In the examples described herein, the input 120 includes at least a biomolecular profile 120a that is associated with a subject, and the output 140 is a skin care prediction for the subject. The biomolecular profile 120a includes one or more “features” that are input into the AI model 100, which predicts the skin care prediction for the subject. The skin care prediction (i.e., output 140) for the subject is therefore the “target” of the AI model 100.

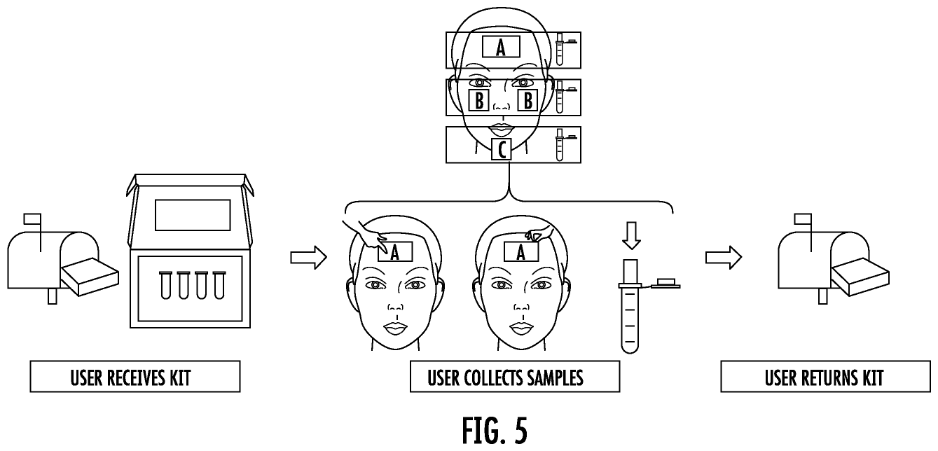

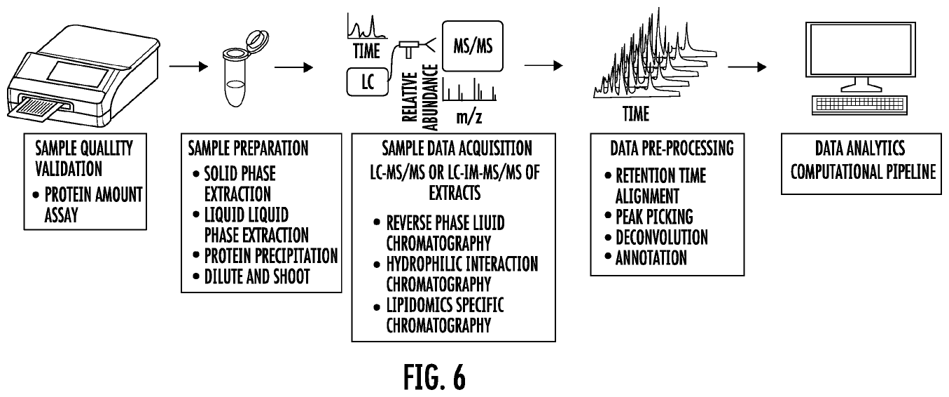

As shown in FIG. 1, the input 120 includes at least a biomolecular profile 120a, which is associated with a subject. The biomolecular profile 120a is obtained from a molecular analysis of a skin sample collected from the subject. Optionally, the skin sample is collected using the sample collection kit (see FIG. 5) described herein. It should be understood that the sample collection kit of FIG. 5 is provided only as an example. The biomolecular profile 120a therefore includes molecular analysis data. In the examples described herein, the molecular analysis is mass spectrometry. Mass spectrometry methods include, but are not limited to, liquid chromatography-mass spectrometry (LC-MS/MS, LC-MS) and liquid chromatography-ion mobility-mass spectrometry (LC-IM-MS/MS). An example mass spectrometry analysis workflow is described with regard to FIG. 6. It should be understood that the mass spectrometry analysis of FIG. 6 is provided only as an example. Additionally, it should be understood that liquid chromatography-mass spectrometry and liquid chromatography-ion mobility-mass spectrometry are provided only as example mass spectrometry methods.

The biomolecular profile 120a therefore is molecular analysis data such as mass spectrometry data. Additionally, the biomolecular profile 120a includes a plurality of biomarkers. Skin biomarkers are molecularly diverse and can include, but are not limited to, amino acids, organic acids, acylcarnitines (e.g., hexadecenoyl carnitine, mass-to-charge ratio (m/z) 398.327), ceramides, fatty acids (e.g., sapienic acid, m/z 254.225), and bile acids (e.g., glycocholic acid, m/z 466.316). It should be understood that the specific biomarkers above are provided only as examples, and biomarkers are therefore not limited to these examples. The presence and level of a biomarker in the mass spectrometry data can be determined by analyzing the data. For example, the presence and level of a biomarker may be associated with a retention time alignment indication of the mass spectrometry data, a peak picking indication of the mass spectrometry data, a deconvolution indication of the mass spectrometry data, or an annotation indication of the mass spectrometry data. It should be understood that the specific characteristics of the mass spectrometry data above are provided only as examples that can be used to assess the presence and level of a biomarker. This disclosure contemplates using other characteristics of the mass spectrometry data to assess the presence and level of a biomarker. Additionally, each of the biomarkers is linked to at least one skin phenotype, skin state and/or disease. Skin phenotypes include, but are not limited to, overall skin health, skin type, structure, function, hydration, sensitivity, age, and appearance. Skin states include, but are not limited to, oily, acne-prone, aging, redness, and dryness. Diseases include, but are not limited to, skin cancer, dermatitis, eczema, and rosacea.

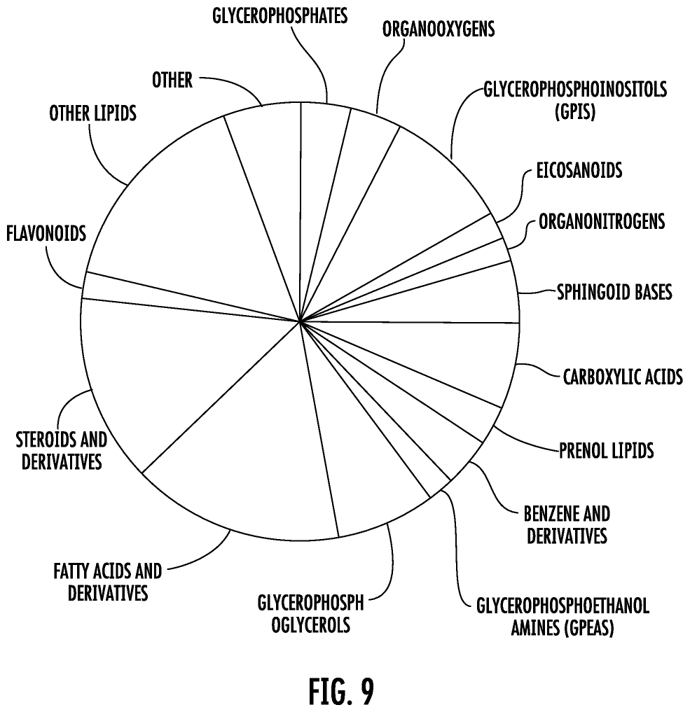

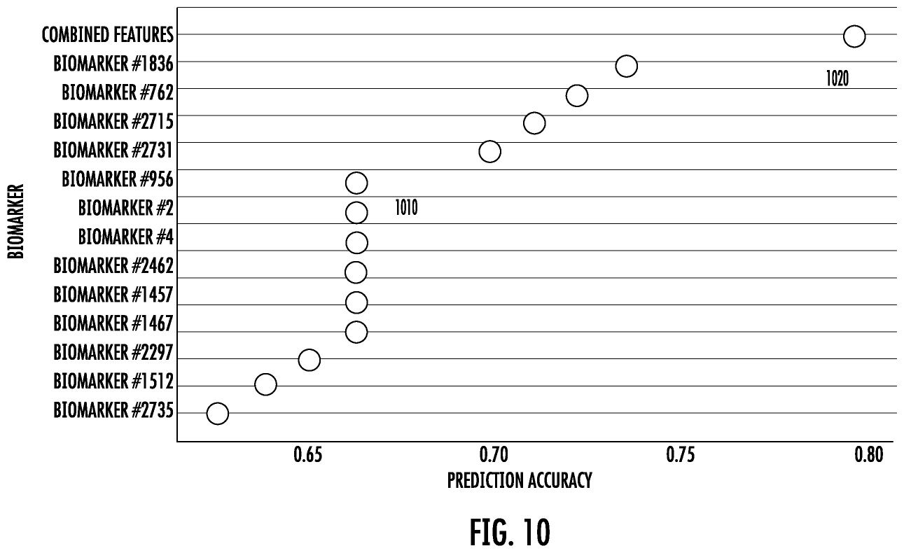

Optionally, in some implementations, a set of predictive biomarkers from the biomolecular profile 120a are selected as features for the input 120. In particular, the top-‘n’ biomarkers predictive of a skin care prediction can be selected, where ‘n’ is an integer. Thus, it should be understood that less than all of the biomarkers present in the biomolecular profile 120a (i.e., only those with relatively higher predictive value) are used as the input 120. The set of predictive biomarkers can be selected from different chemical classes and/or different types of molecules. For example, predictive biomarkers for acne-prone skin are discussed in the examples below. In particular, FIG. 9 illustrates different chemical classes and/or different types of molecules from which predictive biomarkers can be selected. This disclosure contemplates using feature engineering techniques to identify predictive biomarkers among those present in the biomolecular profile 120a. For example, established feature engineering techniques such as Recursive Feature Elimination (RFE) and Random Forest feature importance can be used to identify predictive biomarkers among those present in biomolecular profile 120a. It should be understood that RFE and Random Forest feature importance are provided only as example feature engineering techniques. This disclosure contemplates using other techniques such as Lasso and Ridge regression for feature engineering in this context. Feature engineering, which optionally include supervised, semi-supervised, and unsupervised learning techniques, are known in the art. For example, a skin assessment for acne-prone skin is discussed in the example below with regard to FIGS. 9 and 10. In this example, 16 biomarkers are noted as having high predictive accuracy for acne-prone skin. Therefore, in an implementation where acne-prone skin is of interest, the input 120 may be limited to include these 16 biomarkers in the example. It should be understood that the 16 biomarkers for acne-prone skin described with regard to FIGS. 9 and 10 are provided only as an example. This disclosure contemplates that more or less than these 16 biomarkers (including biomarkers not identified in FIGS. 9 and 10) may have high predictive accuracy for acne-prone skin. Additionally, it should be understood that the number and/or identity of biomarkers among those present in the biomolecular profile 120a may be different for different skin care predictions.

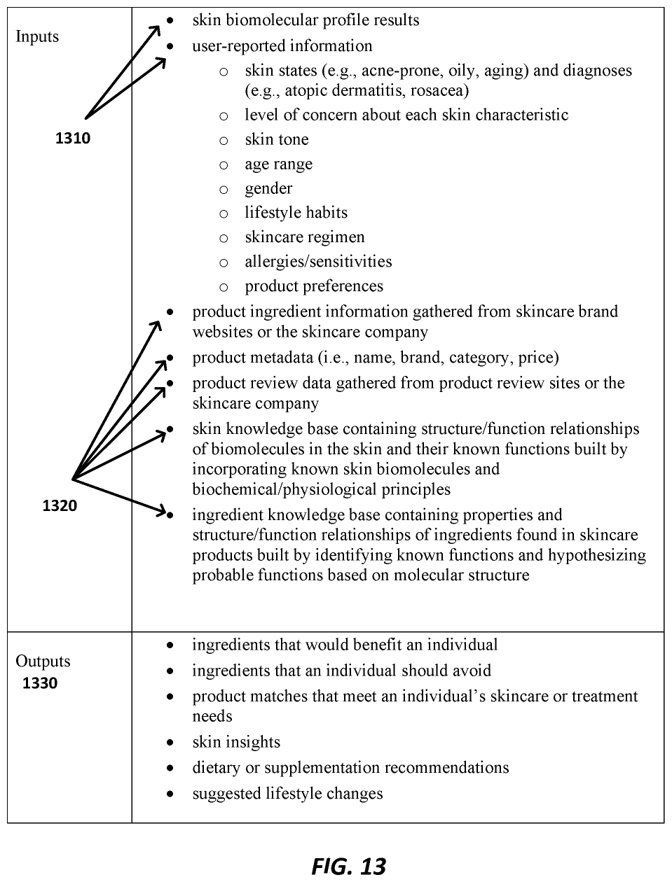

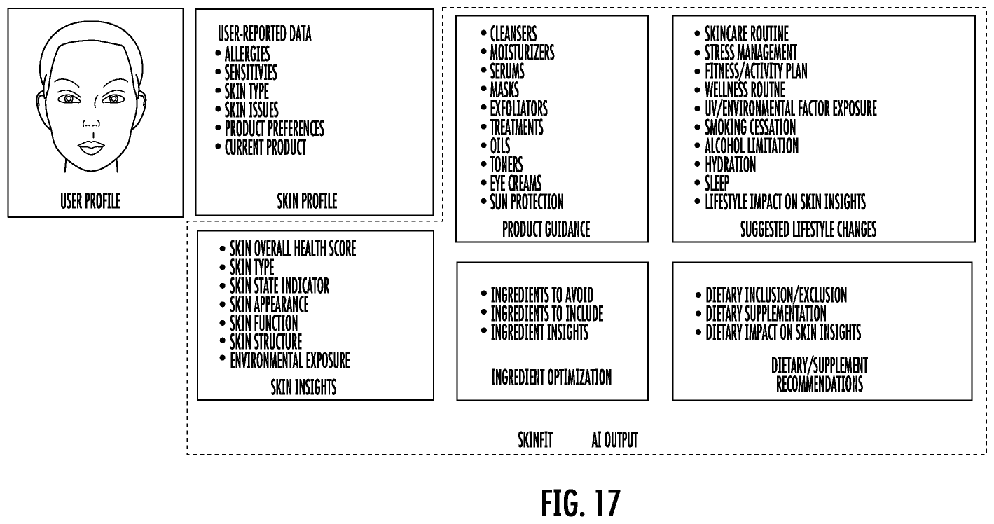

As shown in FIG. 1, the input 120 can optionally further includes user-reported data 120b, which is associated with the subject. In other words, in some implementations, the input 120 optionally includes both the biomolecular profile 120a and user-reported data 120b, and the output 140 is a skin care prediction for the subject. The biomolecular profile 120a and user-reported data 120b includes one or more “features” that are input into the AI model 100, which predicts the skin care prediction for the subject. The skin care prediction (i.e., output 140) for the subject is therefore the “target” of the AI model 100. User-reported data 120b can include, but is not limited to, an allergy, a sensitivity, a skin type, a product/ingredient preference, a product/ingredient usage information, or combinations thereof. It should be understood that the user-reported data examples listed above are provided only as examples. Non-limiting examples of user-reported data are also described with regard to FIGS. 13 and 17. This disclosure contemplates collecting user-reported data 120b from the subject, for example, using surveys. Optionally, a set of features from the user-reported data 120b can be selected as features for the input 120 similarly as described above for biomarkers. In particular, the top-‘m’ features within the user-reported data 120b predictive of a skin care prediction can be selected, where ‘m’ is an integer. Thus, it should be understood that less than all of the features present in the user-reported data 120b (i.e., only those with relatively higher predictive value) are used as the input 120. This disclosure contemplates using feature engineering techniques to identify predictive features from the user-reported data 120b.

As shown in FIG. 1, the AI model 100 is configured to provide output 140 based on the input 120. In particular, the AI model 100 is trained to map the input 120 to the output 140. In other words, the input 120 includes one or more “features” that are input into the AI model 100, which predicts the skin care prediction (i.e., output 140) for the subject. The skin care prediction for the subject is therefore the “target” of the AI model 100. As described herein, the skin care prediction (i.e., output 140) can be a product recommendation 140a, an ingredient recommendation 140b, a supplement recommendation 140c, a dietary recommendation 140d, a lifestyle recommendation 140e, or a skin insight 140f. It should be understood that a product recommendation, an ingredient recommendation, a dietary recommendation, a supplement recommendation, a lifestyle recommendation, or a skin insight are provided only as example skin care predictions. This disclosure contemplates that the skin care prediction may be different than the examples.

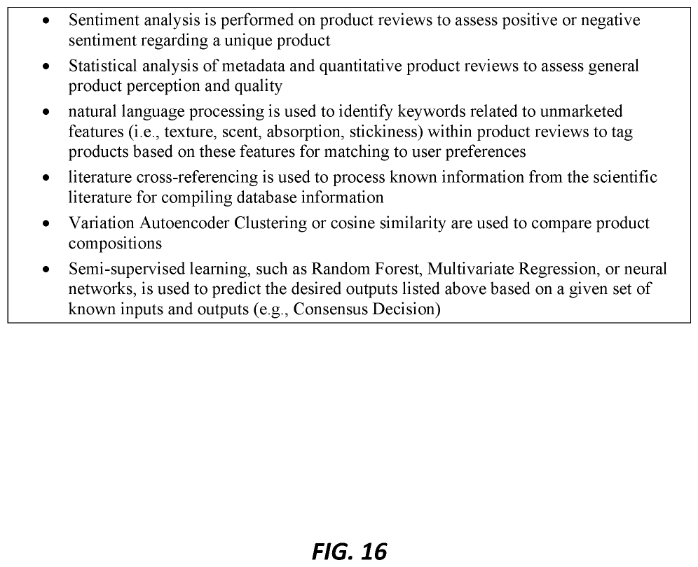

It should be understood that the AI model 100 described above can be trained to predict a particular task. In other words, a plurality of different AI models can be trained to predict different tasks. For example, a respective AI model 100 can be trained for each skin care prediction task shown in FIG. 1, i.e., product recommendation 140a, ingredient recommendation 140b, supplement recommendation 140c, dietary recommendation 140d, lifestyle recommendation 140e, and skin insight 140f. Non-limiting tasks are also described with reference to FIGS. 13 and 16. For example, model training can be conducting using a training dataset including a large and diverse amount of information including, but not limited to, data for a plurality of subjects (e.g., “input” information marked by arrows 1310 in FIG. 13) and data for a plurality of skin conditions, products, and/or ingredients (e.g., “input” information marked by arrows 1320 in FIG. 13). This disclosure contemplates that data can be collected from various sources. Skin biomolecular profile information (e.g., biomarkers) can be mapped to information about various skin profiles, skin conditions, skin diseases, products, and/or ingredients. For example, the information about skin profiles, skin conditions, skin diseases, products, and/or ingredients contains skin structure/function relationships of known biomolecular compounds and molecular class/ingredient properties based on known interactions and structure/function relationships, respectively. Such mapping allows for associations to be formed between a given biomolecular profile, skin state(s) and/or disease(s), and all possible products and/or ingredients with potential for improving a subject's skin. Accordingly, an AI model for a given task can be trained by selecting the appropriate one or more “features” (e.g., from “input” information marked by arrows 1310 in FIG. 13) and “target” (e.g., from “output” 130 labeled 1330 in FIG. 13) in the dataset.

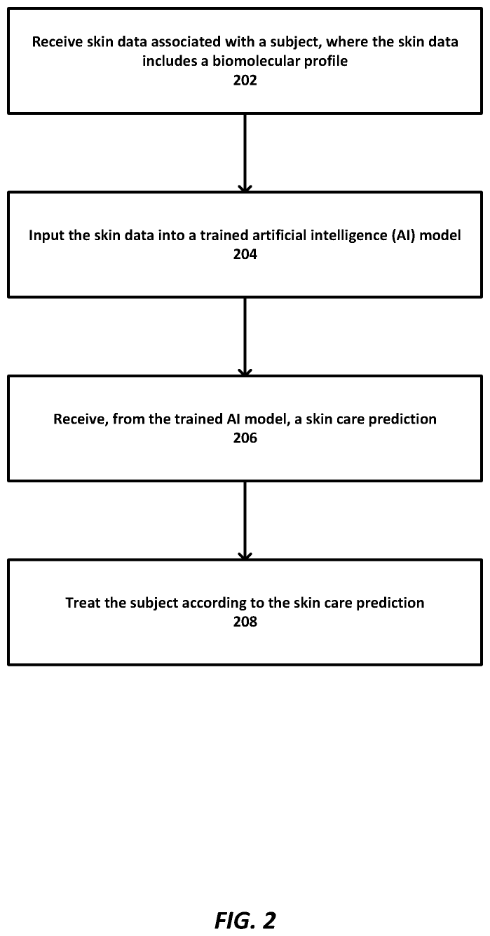

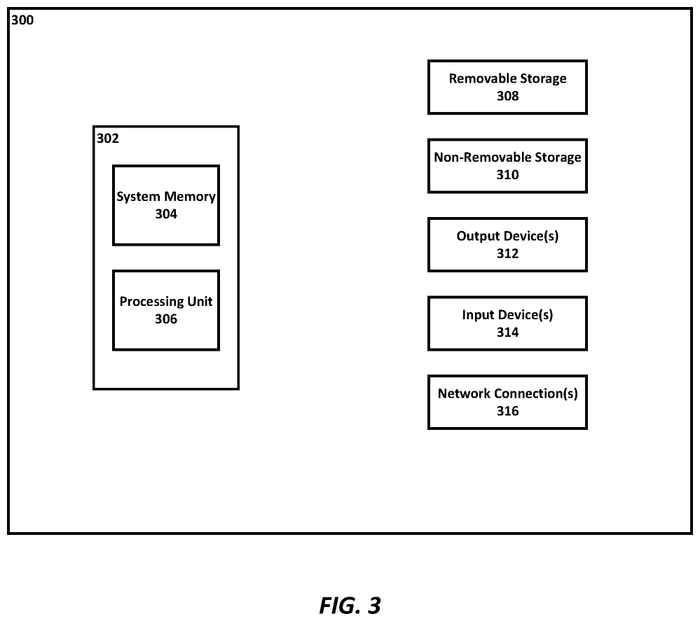

Referring now to FIG. 2, a flowchart illustrating example operations for skin biomolecular profile assessment is shown. It should be understood that the logical operations of FIG. 2 can be performed using a computing device (e.g., the computing device of FIG. 3). At step 202, skin data associated with a subject is received, for example, by the computing device. The skin data includes at least a biomolecular profile. Optionally, the skin data includes both a biomolecular profile and user-reported data. Skin data is described in detail above with regard to FIG. 1.

At step 204, the skin data is input into a trained artificial intelligence (AI) model. It should be understood that the AI model of FIG. 2 can be the AI model described above with regard to FIG. 1. Additionally, at step 206, a skin care prediction is received, for example by the computing device, from the AI model. Optionally, at step 208, the subject is treated according to the skin care prediction output by the AI model. Such treatment can include, but is not limited to, administering to the subject a skin product, a skin ingredient, a food product, etc. Optionally such treatment can include the subject undertaking a change in lifestyle (e.g., avoiding or minimizing time in the sun).

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random-access memory (RAM)), non-volatile (such as read-only memory (ROM), flash memory, etc.), or some combination of the two. This most basic configuration is illustrated in FIG. 3 by box 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

A skin assessment platform is described below that can analyze the molecular makeup of an individual's skin using state-of-the-art mass spectrometry (MS) analysis and a product-skin fit artificial intelligence (AI) that matches individuals to ideal products, ingredients, treatments, lifestyle or dietary changes for the support or treatment of their unique skin.

The molecular analysis, in some embodiments, includes the identification of skin profiles from self-collected user samples and their associated biomarkers that comprise diverse molecular targets (e.g., amino acids, organic acids, acylcarnitines, ceramides, fatty acids, bile acids) using broadscale molecular analysis methods (e.g., state-of-the-art liquid chromatography-mass spectrometry (LC-MS/MS, LC-MS) and liquid chromatography-ion mobility-mass spectrometry (LC-IM-MS/MS)). This data, in addition to user-reported skin-related information, is processed by a big data-powered artificial intelligence pipeline (also referred to herein as “SkinFit AI model”) that uses cutting edge approaches (e.g., deep neural networks, random forest algorithms, and self-attention mapping) to match the unique molecular-level needs of the user's skin, as well as user preferences, to existing ingredients in skincare products, treatments, and lifestyle/dietary changes based on known or unknown biological functions. The system and methods described herein can match an individual's skincare need to beneficial support and treatments, e.g., over-the-counter skincare products (and their ingredients), prescription treatments for skin conditions and diseases, dietary or lifestyle changes/supplementation, through high precision data analytics, analytical measurements, biology (biomarker discovery), and artificial intelligence. Broadscale molecular analyses coupled to advanced data analytics employing machine learning (ML) strategies provide a powerful tool to determine biomarkers, yielding critical information on specific skin states, disease states, nutrition status, skin microbiome, pharmaceutical use, and environmental exposure. The example system and methods can (1) painlessly collect a sample of a user's epidermis, (2) assess the presence and level of biomarkers linked to skin states (e.g., oily, acne-prone, aging, redness, dryness, etc.) or diseases (e.g., atopic dermatitis, rosacea, etc.) using broadscale molecular analyses, and (3) match the needs of the individual's skin to products, treatments, or dietary/lifestyle changes and supplementation based on the biological functions of active ingredients using artificial intelligence, the SkinFit AI model.

A wide variety of skin biomarkers can be analyzed using the methods and systems disclosed herein. Such skin biomarkers are known to those of skill in the art. Examples of skin biomarkers include, but are not limited to, cortisol, fibronectin, Human Serum Albumin (HSA), involucrin, keratin-1, keratin-10, keratin-11, keratin-6, various skin lipids and amino acids, skin PCA, skin lactic acid, IL-1α, IL-1ra, IL-8, and skin histamine. Other skin biomarkers can be found in U.S. Patent Application No. 2019/0369119A1; PCT Application Nos. WO2019026918A1 and WO2018049558A1; and the references Castanedo-Cazares et al. Skin biomarkers for neurodegenerative disease: a future perspective. Neurodegener Dis Manag. 2015 December;5(6):465-7. doi: 10.2217/nmt.15.51. Epub 2015 Nov. 30. PMID: 26619251; and Esteves et al. (2018) Skin Biomarkers for Cystic Fibrosis: A Potential Non-Invasive Approach for Patient Screening. Front. Pediatr. 5:290; which are herein incorporated by reference in their entirety for their disclosure concerning skin biomarkers.

The markers disclosed herein can be compared to a control. The control can be used as a standard of non-diseased, or non-aging, skin. For example, a change in the level of one or more skin biomarkers can be measured in a subject undergoing testing versus a control. The difference between the level of biomarker in the control and in the subject being measured can be given as a fold-increase or decrease.

In the example shown in FIG. 5, sample collection involves a tape-stripping method used previously to assess absorption of molecules into the skin [Rougier, A.; Lotte, C.; Maibach, H. I. In Vivo Percutaneous Penetration of Some Organic Compounds Related to Anatomic Site in Humans: Predictive Assessment by the Stripping Method. Journal of Pharmaceutical Sciences 1987, 76 (6), 451-454. https://doi.org/10.1002/jps.2600760608], drug localization in the skin [Touitou, E.; Meidan, V. M.; Horwitz, E. Methods for Quantitative Determination of Drug Localized in the Skin. Journal of Controlled Release 1998, 56 (1), 7-21. https://doi.org/10.1016/S0168-3659(98)00060-1], and pharmaceutical analysis [Lademann, J.; Jacobi, U.; Surber, C.; Weigmann, H.-J.; Fluhr, J. W. The Tape Stripping Procedure—Evaluation of Some Critical Parameters. European Journal of Pharmaceutics and Biopharmaceutics 2009, 72 (2), 317-323. https://doi.org/10.1016/j.ejpb.2008.08.008]. Kits can be utilized by a skincare professional (e.g., dermatologist, a/esthetician/trained individual) or shipped directly to users for self-sampling. The adhesive substrate painlessly removes layers of the stratum corneum, the outermost portion of the skin, which are returned for broadscale molecular/biomolecular profiling analysis. Samples can be taken from one location on the skin, multiple unique locations (e.g., forehead, chin, and each cheek), or multiple locations pooled together (e.g., one adhesive substrate collects skin from the forehead, chin, and cheeks). The kit can include, but is not limited to, items such as:
Material to remove skin deposits, contaminants, makeup (e.g., alcohol wipe, micellular water wipe, mild face wash);
An adhesive substrate to remove outer layers of skin that covers all or part of the face (e.g., tape, liquid mask that is applied and peeled off);
Labeled adhesive substrates to indicate collection location(s) (e.g., color labeled, numerically labeled, text labeled);
Object to apply even, consistent pressure to the skin during sample collection (e.g., roller, foam block);
Tool for removing the sticky substrate to avoid contamination from hands (e.g., tweezers, tabs, gloves);
Protective enclosure for samples (e.g., zipper bag, plastic sleeve, vial, clamshell);
Soothing material for post sampling (e.g., lotion);
Inserts to hold materials and samples for ease of handling (e.g., cardboard

tube holder, cutouts for each kit material);
Means of safe user viewing during sampling (e.g., reflective film);
Sampling instructions; and/or
Container to hold materials during shipping (e.g., cardboard box, envelope).

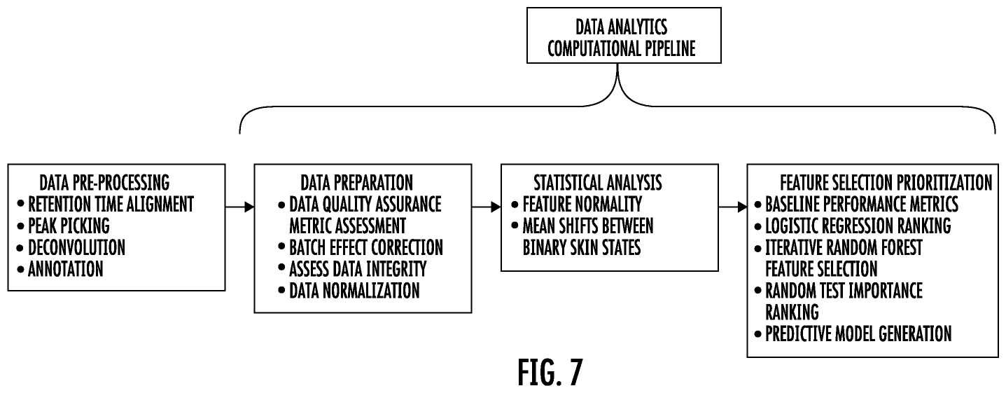

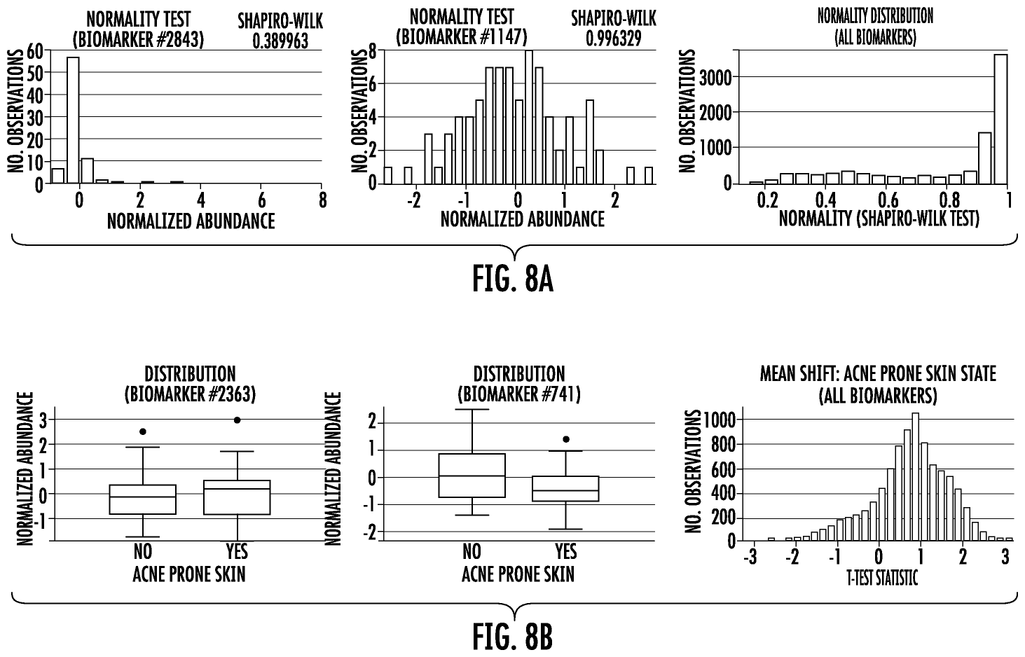

In the example shown in FIG. 6, samples undergo a quality validation (e.g., protein amount assay or similar) and preparation step prior to analysis using LC-MS/MS or LC-IM-MS/MS (or similar) through which the presence and level of biomarkers associated with skin states (e.g., oily, acne-prone, aging, hyperpigmentation) or diseases (e.g., atopic dermatitis, rosacea) are assessed. Data is pre-processed through steps, such as retention time alignment, peak picking, deconvolution, and annotation, before undergoing a data analytics pipeline (FIGS. 7 and 8A-8B).

Each skin state or disease state is described by a unique set of minimum biomarkers. For example, a skin state such as acne-prone skin can be described by 16 biomarkers with high predictive accuracy (FIGS. 9 and 10).

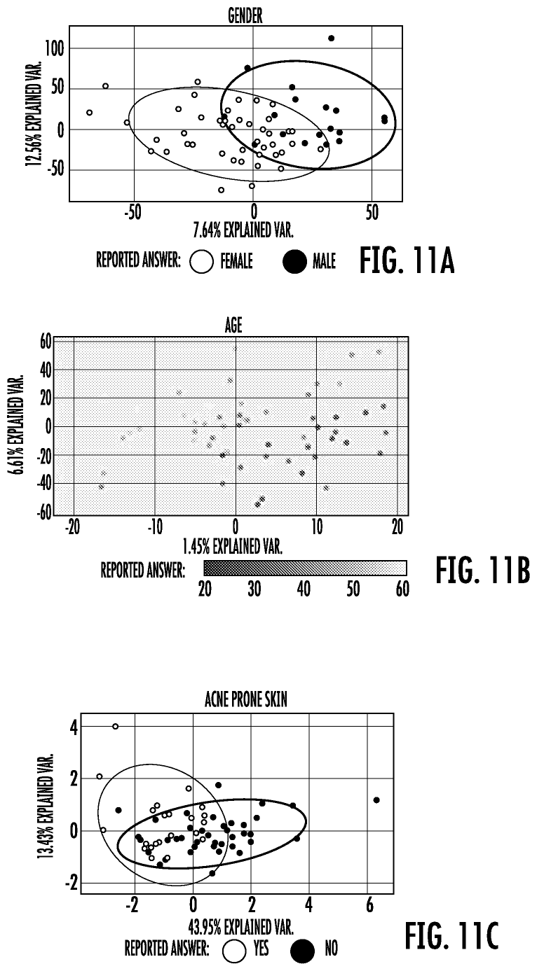

An individual's biomolecular profile may contain markers for one to multiple skin states or disease states. Results of biomolecular profile data can be grouped and analyzed based on data reported by individual users. For example, FIGS. 11A-11C show biomolecular profile data outputs specific to a user-reported data (gender, age, and acne-prone skin state).

Specifically, FIGS. 11A-11C show a high-level overview analysis of biomolecular profile data outputs shown for user reported data for (FIG. 11A) gender, (FIG. 11B) age and (FIG. 11C) skin state (e.g., acne-prone).

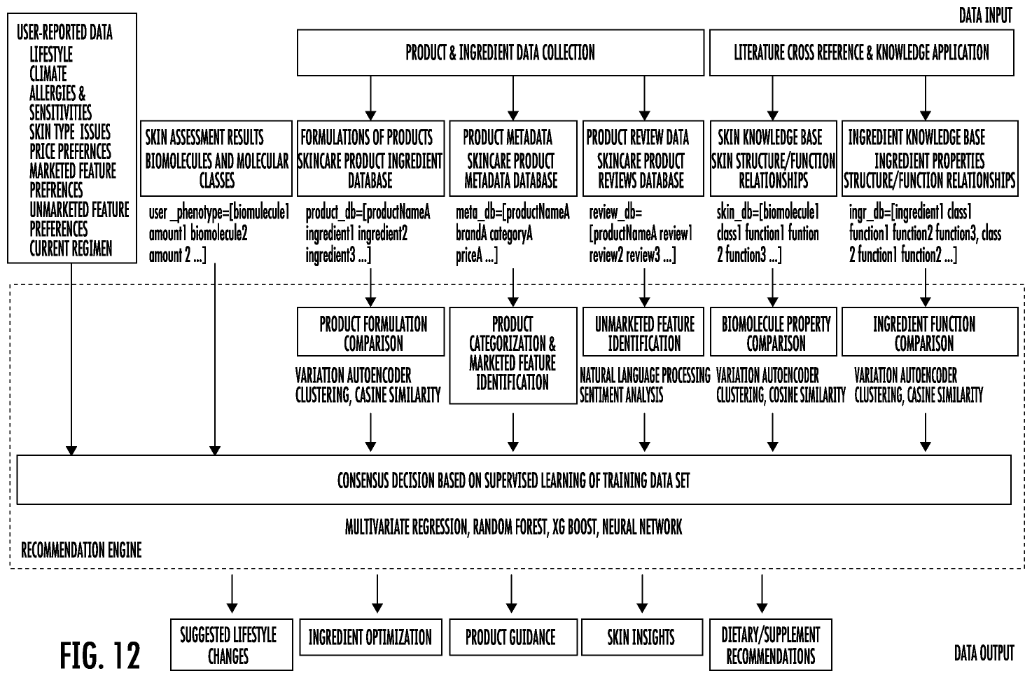

The SkinFit AI model shown in FIG. 12 is configured to convert user-reported data and skin biomolecular profile results into ingredient and/or product (whether over-the-counter or prescription) recommendations, dietary and lifestyle suggestions, and skin insights. The use of “skincare products” below refers to both over-the-counter and prescription products.

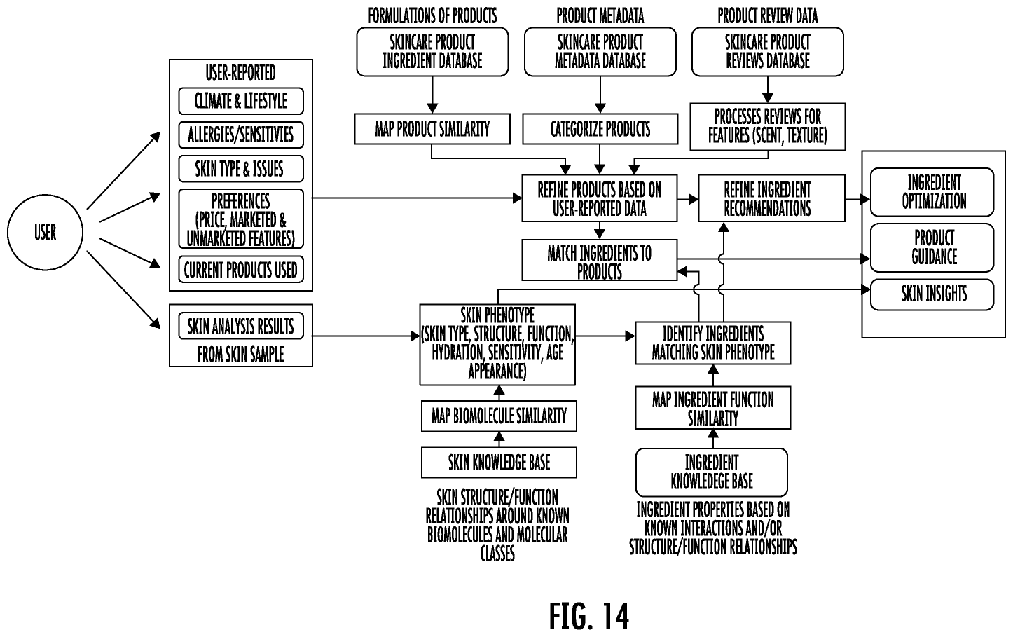

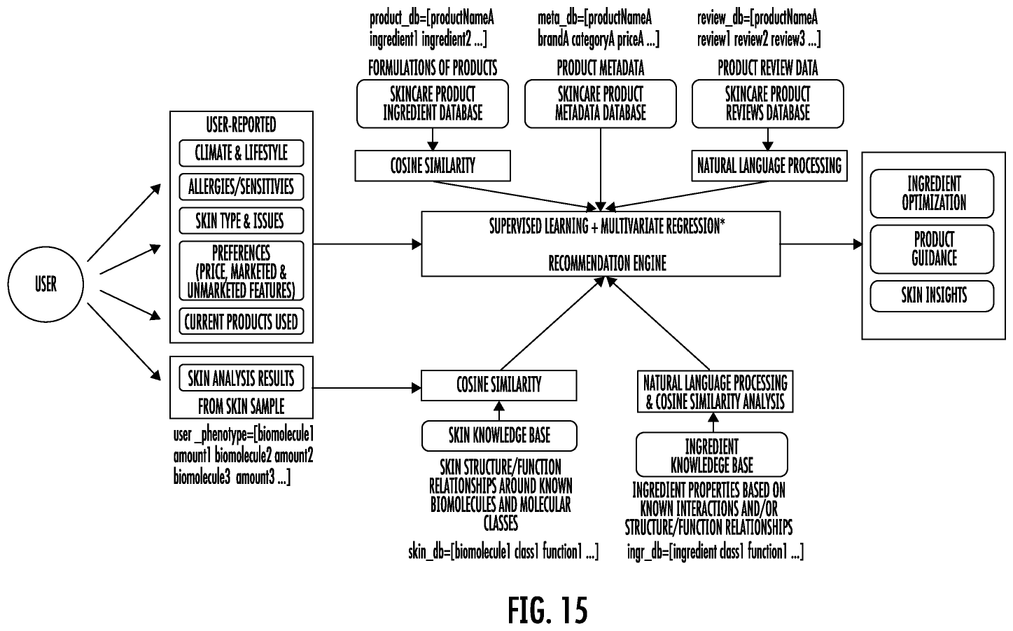

In the example shown in FIGS. 14 and 15, general search and ML techniques (e.g., Gradient Boosted Decision Trees, Transformer Based Natural Language Processing, and K-Means/K-Modes clustering) can be used to parse the variety of data inputs into the desired outputs. In other embodiments, a neural network may be used.

The user's identified skin biomolecular profile can be mapped to a skin knowledge database and ingredient knowledge database, which contain skin structure/function relationships of known biomolecular compounds and molecular class/ingredient properties based on known interactions and structure/function relationships, respectively. This mapping allows for associations to be formed between the user's biomolecular profile, skin state(s) and/or disease(s), and all possible ingredients with potential for improving the user's skin. The list of ingredients is narrowed and optimized by mapping the ingredients to existing products, utilizing product and ingredient metadata, as well as review databases, while accounting for the user-reported product preferences, climate, lifestyle, allergies, and sensitivities.

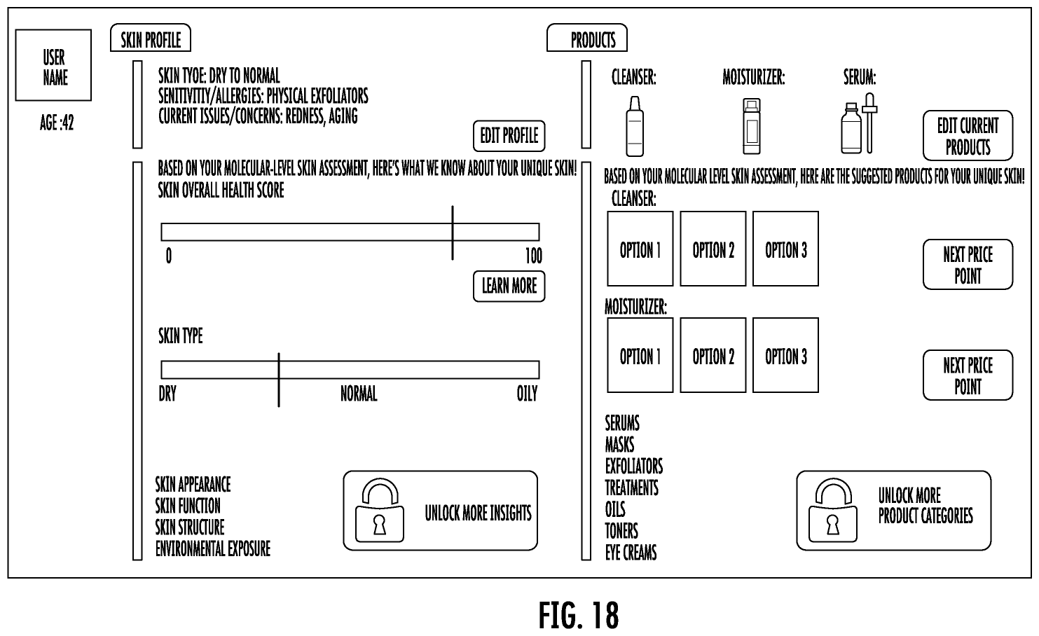

As shown in FIG. 17, the web portal or printed report may include product recommendations with a match accuracy to allow users to make informed decisions about their product selection. Treatment recommendations are delivered, in some embodiments, through a healthcare provider. User-specific skin insights allow for the comparison of user-reported perceived skin states to their scientifically-determined skin biomolecule profile to correct any misconception the user has regarding their skin. Lifestyle, dietary, and supplementation suggestions may provide a means of altering systemically- and physiologically-based skin issues that have limited support with topical products (e.g., vitamin C deficiency). These outputs allow users to find the skincare routine that is optimal for their unique skin, correct any misconceptions they have about their skin. An example report is shown in FIG. 18.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A computer-implemented method for skin profile assessment comprising:
receiving skin data associated with a subject, wherein the skin data comprises a biomolecular profile;
inputting the skin data into a trained artificial intelligence (AI) model; and
receiving, from the trained AI model, a skin care prediction.

2. The computer-implemented method of claim 1, wherein the biomolecular profile comprises molecular analysis data.

3. The computer-implemented method of claim 2, wherein the molecular analysis data is mass spectrometry data.

4. The computer-implemented method of any one of claims 1-3, wherein the biomolecular profile comprises a plurality of biomarkers.

5. The computer-implemented method of claim 4, wherein each of the biomarkers is associated with at least one skin state or at least one disease.

6. The computer-implemented method of claim 4 or 5, further comprising selecting one or more of the biomarkers from the biomolecular profile, wherein the step of inputting the skin data into the trained AI model comprises inputting the selected one or more of the biomarkers into the trained AI model.

7. The computer-implemented method of claim 6, wherein the selected one or more of the biomarkers are the top-n biomarkers predictive of the skin care prediction.

8. The computer-implemented method of any one of claims 1-7, wherein the skin data further comprises user-reported data.

9. The computer-implemented method of claim 8, wherein the user-reported data comprises at least one of an allergy, a sensitivity, a skin type, a product/ingredient preference, or a product/ingredient usage information.

10. The computer-implemented method of any one of claims 1-9, wherein the skin care prediction comprises at least one of a product recommendation, an ingredient recommendation, a dietary recommendation, a lifestyle recommendation, or a skin insight.

11. The computer-implemented method of any one of claims 1-10, wherein the trained AI model is a machine learning model.

12. The computer-implemented method of claim 11, wherein the machine learning model is a supervised machine learning model.

13. The computer-implemented method of claim 11, wherein the machine learning model is a deep learning model.

14. The computer-implemented method of claim 11, wherein the machine learning model is a linear regression model, a decision tree model, a support vector machine (SVM), or an artificial neural network.

15. A method comprising:
obtaining a skin care prediction for a subject using the computer-implemented method of any one of claims 1-14; and
treating the subject according to the skin care prediction.

16. A system for skin profile assessment comprising:
a processor and a memory, the memory having computer-executable instructions stored thereon that, when executed by the processor, cause the processor to:
input skin data associated with a subject into an artificial intelligence (AI) model, wherein the skin data comprises a biomolecular profile; and
receive, from the AI model, a skin care prediction.

17. The system of claim 16, wherein the biomolecular profile comprises molecular analysis data.

18. The system of claim 17, wherein the molecular analysis data is mass spectrometry data.

19. The system of any one of claims 16-18, wherein the biomolecular profile comprises a plurality of biomarkers.

20. The system of claim 19, wherein each of the biomarkers is associated with at least one skin state or at least one disease.

21. The system of claim 19 or 20, wherein the memory has further computer-executable instructions stored thereon that, when executed by the processor, cause the processor to select one or more of the biomarkers from the biomolecular profile, wherein the step of inputting the skin data into the AI model comprises inputting the selected one or more of the biomarkers into the AI model.

22. The system of claim 21, wherein the selected one or more of the biomarkers are the top-n biomarkers predictive of the skin care prediction.

23. The system of any one of claims 16-22, wherein the skin data further comprises user-reported data.

24. The system of claim 23, wherein the user-reported data comprises at least one of an allergy, a sensitivity, a skin type, a product/ingredient preference, or a product/ingredient usage information.

25. The system of any one of claims 16-24, wherein the skin care prediction comprises at least one of a product recommendation, an ingredient recommendation, a dietary recommendation, a lifestyle recommendation, or a skin insight.

26. The system of any one of claims 16-25, wherein the AI model is a machine learning model.

27. The system of claim 26, wherein the machine learning model is a supervised machine learning model.

28. The system of claim 26, wherein the machine learning model is a deep learning model.

29. The system of claim 26, wherein the machine learning model is a linear regression model, a decision tree model, a support vector machine (SVM), or an artificial neural network.

30. A method comprising:
receiving, by one or more processors, a mass spectrometry data set of a subject;
applying, by the one or more processors, the mass spectrometry data set to an analysis employing skin biomolecular profile features derived from one or more trained machine learning models, wherein the skin biomolecular profile features are linked to one of a plurality of skin care or treatment ingredients and/or product; and
outputting, by the one or more processors, an AI-derived output comprising at least one of the plurality of skin care or treatment ingredients and/or products based on the analysis.

31. The method of claim 30, wherein one or more of the skin biomolecular profile features are linked to a skin biomarker that includes at least one of overall skin health score, skin type score, skin structure score, skin function score, skin hydration score, skin sensitivity score, age, and appearance score.

32. The method of claim 30 or 31 further comprising:
performing a mass spectrometry analysis to generate the mass spectrometry data set.

33. The method of claim 32, wherein the mass spectrometry analysis is performed using at least one of liquid chromatography-mass spectrometry analysis and/or laser desorption/ionization mass spectrometry analysis.

34. The method of any one of claims 31-33, wherein the skin biomarker includes one of: amino acids, organic acids, acylcarnitines, ceramides, fatty acids, bile acids.

35. The method of any one of claims 30-34 further comprising:
receiving, by the one or more processors, a request for a sample collection kit through a user portal; and
generating, by the one or more processors, a work order for a shipment to the sample collection kit to an address and user using information associated with a user collected from the user portal.

36. The method of any one of claims 30-35 further comprising:
generating, via a recommendation engine, a first ingredients or a skincare product recommendation using the AI-derived output and a second ingredients or a skincare product recommendation using the AI-derived output in combination with a user provided parameter.

37. The method of any one of claims 30-36 further comprising:
applying, by the one or more processors, the mass spectrometry data set to a second analysis employing skin biomolecular profile features derived from one or more trained machine learning models linked to one of a plurality one of pharmaceutical treatments and/or skin disease/condition states; and
outputting, by the one or more processors, a second AI-derived output comprising at least one of the plurality of pharmaceutical treatments and/or skin disease/condition states based on the second analysis.

38. The method of claim 37, wherein the second AI-derived output includes one of: a skin cancer score, a rosacea score, an eczema score, atopic dermatitis score, and/or a seborrheic dermatitis score.

39. The method of any one of claims 30-38, wherein the skin biomolecular profile features include a first skin biomolecular profile feature associated with a retention time alignment indication of the mass spectrometry data set.

40. The method of any one of claims 30-39, wherein the skin biomolecular profile features include a second skin biomolecular profile feature associated with a peak picking indication of the mass spectrometry data set.

41. The method of any one of claims 30-40, wherein the skin biomolecular profile features include a third skin biomolecular profile feature associated with a deconvolution indication of the mass spectrometry data set.

42. The method of any one of claims 30-41, wherein the skin biomolecular profile features include a fourth skin biomolecular profile feature associated with an annotation indication of the mass spectrometry data set.

43. The method of any one of claims 30-42, wherein the one or more trained machine learning models include one of a regularized linear regression model, a gradient boosted decision tree model, a support vector machine model, and a neural network.

44. The method of any one of claims 30-43 further comprising:
performing, by the one or more processors, a sentiment analysis using product reviews to assess positive or negative sentiment regarding a product; and
performing, by the one or more processors, semi-supervised learning based on the sentiment analysis.

45. The method of any one of claims 30-44 further comprising:
performing, by the one or more processors, statistical analysis of metadata and quantitative product reviews to assess general product perception and quality; and
performing, by the one or more processors, semi-supervised learning based on the statistical analysis.

46. The method of any one of claims 30-45 further comprising:
performing, by the one or more processors, natural language processing analysis to identify keywords related to unmarketed features within product reviews to tag products based on these features for matching to user preferences; and
performing, by the one or more processors, semi-supervised learning based on the natural language processing analysis.

47. The method of any one of claims 30-46 further comprising:
performing, by the one or more processors, variational autoencoder clustering or cosine similarity analysis to compare product compositions; and
performing, by the one or more processors, semi-supervised learning based on the variational autoencoder clustering or cosine similarity analysis.

48. The method of any one of claims 30-47, wherein semi-supervised learning is one of random forest analysis, multivariate regression analysis, or neural network analysis.

49. A system having a processor and a memory having instructions stored thereon, wherein execution of the instructions by the processor cause the processor to perform any of the methods of claims 30-48.

50. A non-transitory computer-readable medium having instructions stored thereon, wherein execution of the instructions by a processor causes the processor to perform any of the methods of claims 30-48.

51. A kit comprising:
an adhesive substrate to collect a sample comprising outer layers of the skin of a user; and
a labeled collection enclosure having a label associated with the user.

52. The kit of claim 51 further comprising:
a second adhesive substrate to collect a second sample of the user, wherein the labeled collection enclosure includes an insert for each of the adhesive substrate and second adhesive substrate.

53. The kit of claim 51 or 52 further comprising:
an applicator configured to apply the adhesive at a consistent pressure.

54. The kit of any one of claims 51-53 further comprising:
a cleaning kit item comprising at least one of: alcohol wipe, micellular water wipe, or mild face wash; and
a substrate removal kit item comprising at least one of tweezers, tabs, or gloves.
